SEQUENCE LISTING (5 sequences)

SEQ ID NO: 1 - DNA, 3778 bp, artificial sequence
  Synthetic
catcattaaacttctgacaagcctttatgttaatgcacttagtaattcccagactgtagagtaaaaccataaatgtttttctgggaaatacacttttttttttggccacacccatggcatgtgggagttcctgggccagggattgaatccgcactatagcagtgaccagagccactgcagttgtcaacactggctatgccacaagagaactccctgggaaatatgctttatattttcaaagttgttttgaaacacccccctggcatttgtaagagaagtgagagaagttccttgctcccttcggttatagaaatataaggccctcatccactataaagaacagggggaaggaaaaagataagtcttgtgaaagagctgcactggccacctaaattgactcatgtgagaaactgctttcaaatacggaacacctaagacaaagccaacaagctggtggaccacaccctcccaattaactagaagggattctctactcactcattctgatgacacagtccattcttcatgaatgcagccaaggctaggcccccatcagtcacaccacagtgatggttcctatggctggtgaaccttccttctctggggctttggaaagattgcagtcactgacactggagaaggaatataggttctcaaaacttcttgagtacctattaactcattcaatactgaccttgagcacagttgattagactcagttgattctcattattcatagcagttgtgttctatcaaatcactgccaacactgaatgaatgatggatcactgagccattgctcctaggggaagtacaaagttaggttcctatcagcctctggtcacattttcaccaaccaatcaatatataaccttgttttatgttcatttctgttttaaaatatttatttaatatatattattgatttattaacattgtattcataggcaaacacactataactcaagcctgaattatgcttacataatacacatatttttccattaaggaaattattttccctaagtcatagcacaaccttcttttttttaaattgtggtaaaatatacataagacagttcagtgtacagttcagtggcattaaatacatttatattgttgtgcaatcatgatcaatatccatctccagaactttttgaccttcccaaattgaaaattttgtatctattgaacagtaggtttcccccatcccaccccacctctcctccagcacctggtaaccaccattctcctttccatcacagccttcctgtatgtaggagcacagcactatgctttgggaccattttaaacagcagaatcatcccctaaaaaaacaagtaaaaatgtgaaaaacatggtactatacagattgcaaagagggcacttgcttaaatctgagagctgaaacaaggcagagaattgccttgtttgacctcaactgggaccctgtgtcctcaagatttttgctgttttgtacaggcatttgaatgactatcaaagtgccatgagtattgattctggagttaaaaataaatttcagcaagtagacaaatttgcatacagggaatccatgaataacgagcattagctgtacggaaatgacagctttgtttcctttactccaggtactaagaataaagagctgaaatgtggcttgtgatagggttgtcttggaatatttcacttgcctcagaggcctagagagctagcctatgtgaactgataattggcagctacaacctgaagcagttctagttcatgtggaggataaacttaagcattatctcaaacccctctgcatgaaacaaagaccaaacattcaagtactagttatcaatcacttactatgtgacaggcactatactcagcaatttacatgcattattgaattacatcccccaaaactgaaggttagagaagctaagtatctcatccattattacactgttagaagtggcaaagttgagatttgaactcaggtctatctgactccaaagcctatttcccaacagctttgcaattctattcaagtttaaaaaaaaaaatatctgatttactcagaagtgtataggagcatatgttatgattattataatattacaaagatttatatgttgaaaaataaatttaccaaaaaaaccctttataagcctgatctaatattgctccacaacaaagaatttctgaaatccttcagggcatctggtttgtgtctgtttttccttaatctttaatgatgagcaagtctaatgcattatgtaaggccatttttctcaagagatgtagatacctcctaagaatttgatgaaaatgcattaacttttcaggctactgagttgcattttagtgcactgaggcagtaaatgagtgtacaatgtgcaaaagtagtgacctaaaaaataaatatttgatatgaaccactgcattctcttggaaaaaaaaaaaagtaatggattaactctcttaggagtccttagcttccccaaaaggagtaggaagaataaatctcctgtggcctggaaacagcttctgtttctcactggctatgtttgtttagctctttaatagttcatttgattagatcttgtggctcctaaagctaaggttgagagtttgagctctacagaggccacttaaatttagagaacaaaaagctctattctctgctcccagaccttaccccaaatccctgccaggtgtctgccctctggtcaaatgagaaactggcaaaggggtgcaaacctagcacagaattgggaaacagaaaaatgggcaccctttattatggtgctccttctcttttatgtgtttacaatacttgggcataatttacagagaatagatactacattttttactttcaccactggaaatctgaggcaaactgcattatcagtcataaaattcattatctttctattctaagttattctaagcttattctaagctcagatagctgacattatcctcttggtaataaacaatgaaaaaacacatcttctgagcaatattaatctgcaactttaggataggagaaaatcagttgaaaactgagcacgattttcacgtgaataaaagatattatttaaaaataattccatgtgtaatataacagaataagtatgattttcattatgtactagaaatttagtcaggaaaacaagtttctcaaattatagctgaatatattttactagtatcacaatcttaaattttaattcaggtcttcctaatttaaatctgtatttctctgattacacaggactaaaaataatttaaaacagcaaataaaattcttttttcctcaaatgtttgtctaaataatgtaaaatcattttatttttttgaggaaaaagacatttcaactttttaagtatgaagtgtaaaagaattacttatttaaattacaattttaaagtttcactaataaagattaataatatttaagtgcagtttatattattgttaacatagattttaatttttcaaatgtcacatatatctttcattatttgtagatttatttcttttatgaagtagtcaaatgaatcagctcacccttgactgtaacaaaatactgtttggtgacttgtgacagacagggttttaacctctgacagcgagattcattgtggagcaagagccaatcatagatcctgacgacacttgtctcatcaagtggaatataaaaagccacttggaatacagtataaaagattcactggtgtggcaagttgtctctcagacagtgcaggcattaaaattttgcttggcg

SEQ ID NO: 2 - DNA, 4994 bp, artificial sequence
  Synthetic
ttactcaaaagcaaaagtaaaaggaagaaataagaacaaggagaaagattgtattgattttaaaatcatgcaaaaactgcaaatctatgtttatatttacctgtttatgctgattgttgctggtcccgtggatctgaatgagaacagcgagcaaaaggaaaatgtggaaaaagaggggctgtgtaatgcatgtatgtggagacaaaacactaaatcttcaagactagaagccataaaaattcaaatcctcagtaaacttcgcctggaaacagctcctaacattagcaaagatgctataagacaacttttgcccaaagctcctccactccgggaactgattgatcagtacgatgtccagagagatgacagcagtgatggctccttggaagatgatgattatcacgctacgacggaaacgatcattaccatgcctacagagtgtaagtagtcctattagtgtatatcaacaattctgctgactgttgttccagtgtttatgagaaacagatctattttcaggctcttttaacaagctgttggcttgtacgtaagtaggagggaaaagagtttcttttttcaagatttcatgagaaataaactaatgagactgaaagctgctgtattattgttttccttagctaaacagctgaaaataaaaaataaaatgcttgcatagcattgttatatagtttaataagacaaatataacatgcttatgctttcacagcttaatgccaccaaggcaaggattgggagtttctacaagcaatgtggaaaaaaaggttggttgtctgaaataggcatttgtaataacaggtttttttcactaatgataaagaagggaagatgtaaatttgcagatattgagccccattggggcatttgctgcacccctagaatgacttctgttattcagaacgatttctcacagtgtttctatgttcttcacaaattaaaatgtctaattttgaaagctattacactggaaagtataaaaaatatttttaaaaaatttaatgttttggtaagagcaatgatgaagtaaacatagcataatggtaattatgagctaattatcagaaaatgccaagaaataaacattttatcaagtaggttatggctcacaaagtcctgcttataccttgaccatggtactattgttgagagtaccctgtctgaatatttccaggcaggcacatgcttaataagctctacaatattattttctttttcataggagggagaaagaactattacctgtagtatccacattgcttatgaaggacaatatatttcataccattcctattacaatcagttcaaaagtatacacaaggaaagggagacaggcaccttaacagagaaggcatgacaagaaagatttttgtgccatgtgtctgtgattttgctttatacagtgctttacccactttaaactagactcaaaacagtttcaaaatattattcttcttattaagtaattaggctataatgcaacaaataatttttcttgaaaactatgctatcagataatcctagagtagatttgccttatttataaacaatcttggaaaaccaaaaggaaagctgtttctaaatgcttctgcttacaatgacagcatggccttaacaatgttttctaagttttgagatagcctgaatgcaacatttaaattctggtgctaagtgccttctagtttggttcctttaaaaaagctatcccaggccaaaacataacagatgtactatattttctactaattcccgaggctcagttagttgctcagtgtgtctcgtccccaggtaattcaggcctgggggaagggttccttcttccagactgattggtacagctgctcagtaagtgtaactactcagattcccaaagaattctaagtggatgttcctccacagtgtctcttgttctctctaatcatcatcattttaaaatttcatccactcttcattcctttacagaattttctttagtctacagttttctagaaaggaagtaggtttctcataaacagctgaaaaaacatattgaaaaaaatctgaaaagctatagtaattatttcatttgatatttttctgaattatgaatgaaattctacagtttttcattttaaaagactaaatatgcatgcactattccaatagaaaaaaagctcactgattaatatgaaggagtttgttcatttttcatgaaacaatttcaataactcttttctttttttactcatttttagctgatcttctaatgcaagtggaaggaaaacccaaatgctgcttctttaaatttagctctaaaatacaatacaataaagtagtaaaggcccaactgtggatatatctgagacccgtcaagactcctacaacagtgtttgtgcaaatcctgagactcatcaaacccatgaaagacggtacaaggtatactggaatccgatctctgaaacttgacatgaacccaggcactggtatttggcagagcattgatgtgaagacagtgttgcaaaattggctcaaacaacctgaatccaacttaggcattgaaatcaaagctttagatgagaatggtcatgatcttgctgtaaccttcccaggaccaggagaagatgggctggtaagagtttactgaaaataacactcttaaaatcttgttatgtttttattcataatgtgaatgagtagtagtggaaaataactaccagtttcctaagctagacaaaagtatcttaccccaatggtagccctgtacccaataaaagtaggtgttcagtttcatatcctatgaaataccctcttgatacttttactttgcatgaggatttagaagaaaaaagttttactataatccttaacttaggaaattcttttgaattggaaatgaaacacaaattgcttttcattgatatgccatatgattatatgaataaaacatgaaatcttcatattggattctagtatatacccaagtaaatattttttccctagaagagtgccaagtgtgttaaaaccttttggtttaataaagcagaaaaaaataaactctaaaaatcataattaaaaatgaaatgcttttatttatagcaattaactacaacatgtttagacttacatactattaaatataatatatttaagatcccctcatgataaatatgttcattattttgtaggctgttgatgcactaatatgtatgtagattactttgtgaattgcccttaataaaatttaaaactttaggctagtaaacctgtaacactcaacttagttctgaactatctcactattcttttgcaagaatttacttaggtaatgccaactaatttattccaaggccaaaaagatgacaatgtcttatatattataaaaactaataaaaaccattttaaaacctagtataaatttaaaggtacttgctcttctggttcatctcttcttttgtttacttctgctttcaaaaacttatttattgtgaccatattctttacttccatttattgttataatttataagatactatacttgcaagcaataaatgttatctttttagcttttaaatggtctcatttgaaaagaatatataattagtaagtcatagctactttaaataaaaacttattctttaagagattaaacacttctccaagtgatctgtttttctttaattaaaacgttattaactcccaaaatgatgttattgtttttttataatcttaaataccaataattaccaggtctattttgattttgatacaggataaaaactactattaattacttaagaatgtgttcttttttatatgtaccattttcatgatcaaagttggtgatatgactgaggttttgattattattaaacagatagttaatatgatatattcctcatttttccaaatgaaaggaaaaatgtcttatatggaggaaaagattggggcagggggattagtaaattattacttaaatatctgaataggaggatttttcaatgaaaggataaaggaagaatgattgtatcatctgaatctttccctccctttcctggagtttgtcctttcaacccagtatacctaccactcccttcatcacctactttcccattacagtccctatgtgttgggtggtaactattttgttttggtgttaatatccaagtttcccttaataacacctagtgaatggaggaaggatgagcatacctacccatcagacatatttagccaccatatttaatcaacaagcatgaagaaaggaagctagcctctccccttcctttcctcctgcctctctctctcttctctgtcctcgctccctttcttcccatcaatattttcagagcacctcttatgcgccaggcattgggatactcaaactggaggaaacaagaaaaaaaaaaaaaaaaggcgaagacctcagggaaatttatattgctgctatatttttttgagcctagtgtaaattaaaattccttaatgctgtgccttttaaaaacacaaataagcaaaatagtttatttcttcaacagttaaatccttagggtaggaaagtgattcaggatctattgctaactattaactcttctttcattttcacacagaatccctttttagaagtcaaggtaacagacacaccaaaaagatccaggagagattttggactcgactgtgatgagcactcaacagaatctcgatgctgtcgttaccctctaactgtggattttgaagcttttggatgggactggattattgcacccaaaagatataaggccaattactgctctggagagtgtgaatttgtatttttacaaaaataccctcacactcatcttgtgcaccaagcaaaccccagaggttcagcaggcccctgctgtactcccacaaagatgtctccaatcaatatgctatattttaatggcaaagaacaaataatatatgggaaaattccagccatggtagtagatcgctgtgggtgctcatgagatttatattt

SEQ ID NO: 3 - DNA, 1446 bp, artificial sequence
  Synthetic
ggttcattacttcctaaaacatggaaggtttttcccctcaataattttgaaactgtgaaattatgtaccacaggctacatgcctggagtatgctacagtcacttaagcacaagcgacagtatatgaactaaaagagagaatatatgcaatggttggcatttaaccatcaaaacaaatcatacaataaaaagtttttatgatttccagagtttttgaacgaggagatcaaattccatttatgttcatatatattacaacatatgcaggtaaatgaaagcaattctccttgtgttctggtgaattaaaggagtatgctttaaagtctatttctttacaattttacttaatatttacagaaaaatctatatgtagtattgataagatgtaggattgttatataccattatttgagtcatccttaaacacttgaattatattgtatgatagcatacttggtaagagaagattccacaaaaatagggatggtacaccatatgcaagttcccattcctattctgattgatacagtacattgacaattcatgccaatggtgctaattcaataggctgaatggctggtgttatcaggtttatcaaataaaagcattcagtaaagtaatggttctcctttcttcaggtgcattttcatactctttcaaatggagaatggattttctttaatgaaagaaaaatcatttttctagagctctgcattcaattctgtagcatacttggagaaactgcatttaaaaggcagccaaaaagtattcatttttatcaaaatttcaaaattatagcctgcctttgcaacactgcagtttttatgataaaatcatggcaatgactgattctatcaatattgtataaaaagactttgaaacaattgcatttatataatatgtaaacaatattgttttgtaaataaatgtctccttttttatttactttggtatatttttatgtaaggatatttcaaattaagtattaaggcacaaatacatgtcatgtgacagaaaagcaaatgcttatatttcggagcaaattagctgattaaatagtggtcttaaaactccatatgctaatggttagatggttatattacaatcattttatatttttttacattattaacattcactatggattcatgatggctgtataatgtgaatgtgaaatttcaatggtttactgtcaatgtattcaaatctcaacgttccattattttaatacttataagtaagcataccaaaatgatttaactcaattatctgaaatcagaataataaactgatgatatcttaagaattgttaatttaattttataattcgataatgaatatatttctccatatatttacttctattttgtaaattaggattttgttaatcaaatacattgtacttatgactaagtgaaattatttcttacatctaatgtgtagaaac

SEQ ID NO: 4 - DNA, 720 bp, artificial sequence
  Synthetic
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 5 - protein, 239 aa, artificial sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

(19) United States
(12) Patent Application Publication
(10) Pub. No.: US 2014/0223592 A1
(43) Pub. Date: Aug. 7, 2014

(54) PIG MYOSTATIN GENE LOCUS AND USES THEREOF

(71) Applicants: Yanzhen Bi, Wuhan (CN); Xinmin Zheng, Wuhan (CN); Xianfeng Qiao, Wuhan (CN); Ximei Liu, Wuhan (CN); Liping Zhang, Wuhan (CN); Wenjun Hua, Wuhan (CN); Li Li, Wuhan (CN); Hongwei Xiao, Wuhan (CN); Jingrong Zhou, Wuhan (CN); Qingxin Wei, Wuhan (CN)

(72) Inventors: Yanzhen Bi, Wuhan (CN); Xinmin Zheng, Wuhan (CN); Xianfeng Qiao, Wuhan (CN); Ximei Liu, Wuhan (CN); Liping Zhang, Wuhan (CN); Wenjun Hua, Wuhan (CN); Li Li, Wuhan (CN); Hongwei Xiao, Wuhan (CN); Jingrong Zhou, Wuhan (CN); Qingxin Wei, Wuhan (CN)

(73) Assignee: PIG MYOSTATIN GENE LOCUS AND USES THEREOF, wuhan, Hubei (CN)

(21) Appl. No.: 14/128,995

(22) PCT Filed: Jul. 6, 2011
(86) PCT No.: PCT/CN2011/001119

§ 371 Date: Apr. 11, 2014

(51) Int. Cl. C12N 15/85; A01K 67/027

(52) U.S. Cl. CPC C12N 15/8509; A01K 67/0275
USPC 800/21; 536/23.1; 435/320.1; 435/252.33; 800/13; 435/440

(57) ABSTRACT

A pig myostatin gene locus and uses thereof are provided. Also provided includes an expression cassette comprising a promoter, a foreign gene and a following terminator; the promoter is a DNA molecule as set forth in any of 1)-4): 1) nucleotides at positions 2642-3778 starting from the 5′ end of SEQ ID NO. 1 in the sequence listing; 2) nucleotides as set forth in SEQ ID NO. 1 in the sequence listing; 3) a DNA molecule, hybridizing and having the same function with the DNA sequence as defined in 1) or 2) under stringent condition. Experiments show that the pig myostatin gene locus provides a valuable gene source for gene targeting, as well as introducing and expressing a foreign gene at this site.

### TECHNICAL FIELD

The present invention relates to the field of biotechnology, particularly to a pig myostatin gene locus and uses thereof.

### BACKGROUND OF THE INVENTION

In the past decade, the rapid development and wide application of transgenic technology, especially the combination of gene targeting and somatic cell cloning techniques, enables the realization of gene site-directed modification of transgenic animals. The key of the technology is that a foreign gene is site-directedly integrated in a specific location in of the genome of a transgenic animal and stably expressed. This “specific location” must meet the following requirements: (1) the deletion or mutation of gene sequence in this specific location does not cause the death of the host animal; (2) the deletion or mutation of gene sequence in this specific location does not cause abnormality or malformation in growth and development of the host animal; (3) the deletion or mutation of gene sequence in this specific location does not cause infertility in the host animal; (4) gene sequence in this specific location is less impacted by the level of DNA methylation, especially the absence of imprinting modifications, which ensure that a foreign gene can be effectively expressed. Therefore, finding an ideal “specific location” satisfying the above conditions is a prerequisite for the successful application of gene targeting and somatic cell cloning.

Myostatin gene was originally cloned from a cDNA library of mouse muscle tissue by McPherron et al. in 1997. This gene, as a member of TGF-β family, is a transforming growth factor. As demonstrated by gene knock-out experiment, inactivation of this gene results in mouse muscle tissue proliferation and body weight gain; and the mouse can normally survive with fertility. Subsequently, it is found in animals such as bovine, sheep etc. that myostatin gene primarily functions to negatively regulate the growth and development of muscle; in addition, inactivation of myostatin dose not results in physiological disorder in the above described animals.

### DISCLOSURE OF THE INVENTION

One of the objects of the present invention is to provide an expression cassette.

The expression cassette provided by the present invention comprises a promoter, a foreign gene and the following terminator:

The promoter is a DNA molecule of any of 1)-4):

1) nucleotides at positions 2642-3778 starting from the 5′ end of SEQ ID NO. 1 in the sequence listing;
2) nucleotides as set forth in SEQ ID NO. 1 in the sequence listing;
3) a DNA molecule, hybridizing and having the same function with the DNA sequence as defined in 1) or 2) under stringent conditions;
4) a DNA molecule, having at least 70%, at least 75%, at least 80%, at least 85%, at least 90%, at least 95%, at least 96%, at least 97%, at least 98% or at least 99% homology as well as the same function with the DNA molecule sequence as defined in 1) or 2);

The terminator has a nucleotide sequence as set forth in SEQ ID NO. 3 in the sequence listing.

The stringent conditions may be: hybridizing at 65° C. in a 6×SSC, 0.5% SDS solution, followed by washing the membrane with 2×SSC, 0.1% SDS and 1×SSC, 0.1% SDS, respectively, each for one time.

The foreign gene may be a pig myostatin gene or a green fluorescent protein-encoding gene;

The pig myostatin gene has a nucleotide sequence as ser forth in SEQ ID NO. 2 in the sequence listing;

The green fluorescent protein has an amino acid sequence as set forth in SEQ ID NO. 5 in the sequence listing;

The green fluorescent protein-encoding gene has a nucleotide sequence as set forth in SEQ ID NO. 4 in the sequence listing.

A recombinant vector, a recombinant strain, a transgenic cell line, a transgenic animal embryo or a transgenic animal containing the expression cassette each falls into the protection scope of the present invention.

The recombinant vector may be obtained by inserting the expression cassette into the pUC19 vector between the KpnI and HindIII restriction sites;

The transgenic cell line may be obtained by introducing the recombinant vector into a host cell, which, specifically, is a C2C12 cell;

Another object of the present invention is to provide a terminator.

The terminator provided by the present invention is a DNA molecule of any of 1)-3) below:

1) a DNA molecule as set forth in SEQ ID NO. 3 in the sequence listing;
2) a DNA molecule, hybridizing and having the same function with the DNA sequence as defined in 1) under stringent conditions;
3) a DNA molecule, having at least 70%, at least 75%, at least 80%, at least 85%, at least 90%, at least 95%, at least 96%, at least 97%, at least 98% or at least 99% homology as well as the same function with the DNA sequence as defined in 1).

Use of the terminator in terminating the expression of a foreign target gene also falls into the protection scope of the present invention.

Unless specifically indicated or individually defined, the scientific and technical terms used herein have undoubetedly same meaning as commonly known by the skilled in the art to which the present invention pertains. Furthermore, the materials, methods and embodiments described herein are intended to be descriptive and illustrative, but not to limit or define.

### BRIEF DESCRIPTION OF THE DRAWINGS

FIG. 1 shows amplification of 5′-terminal sequence of pig myostatin gene locus;

FIG. 2 shows 5′-terminal sequence cloning of pig myostatin gene locus;

FIG. 3 shows detection of transcriptional activity in 5′-terminal sequence of pig myostatin gene locus;

FIG. 4 shows amplification of 3′-terminal sequence of pig myostatin gene locus;

FIG. 5 shows 3′-terminal sequence cloning of pig myostatin gene locus;

FIG. 6 shows detection of transcriptional activity in 3′-terminal sequence of pig myostatin gene locus;

FIG. 7 is an electrophoresis photograph of the PCR product amplified from the coding region of pig myostatin gene;

FIG. 8 is a restriction map of vector pUC19-3 of pig myostatin gene locus;

FIG. 9 is a restriction map of vector pUC19-53 of pig myostatin gene locus;

FIG. 10 is a restriction map of vector pUC19-5MSTN3 of pig myostatin gene locus;

FIG. 11 is a schematic diagram showing the structure of pig myostatin gene locus;

FIG. 12 is an electrophoresis photograph of the PCR product amplified from the coding region of green fluorescent protein;

FIG. 13 is a restriction map of pUC19-5EGFP3 vector;

FIG. 14 shows identification of the expression of pUC19-5EGFP3 vector;

FIG. 15 shows a relative expression intensity of pUC19-5EGFP3 green fluorescent protein;

### BEST MODE FOR CARRYING OUT THE INVENTION

Each of the experimental methods used in the following examples is a conventional method, unless otherwise indicated.

All of the materials, reagents and the like used in the following examples are commercially available, unless otherwise indicated.

The present invention will be further described below in conjunction with specific examples. It should be understood that these examples are presented only for the description of the present invention, which, however, are not intended to limit the protection scope of the present invention. The specific experimental conditions and methods are not indicated in the following examples, which, usually are performed according to conventional conditions, such as conditions recommended by Molecular cloning experimental guide, J. Sambrook, DW Russell et al., 3^{th} edition, Science Press, 2002 (translated by Huang Peitang et al.).

### Example 1

### Cloning and Activity Identification of Regulatory Regions of Pig Myostatin Gene Locus

I. Cloning and Activity Verification of the 5′ Untranslated Region of Pig Myostatin Gene Locus

### 1. Cloning of the 5′ Untranslated Region of Pig Myostatin Gene Locus

TABLE 1
Experimental materials

| Main materials | Sources | Instructions |
|---|---|---|
| Ear tissue of hubei | Newborn piglets from the | quick-frozen in liquid |
| white pig | experimental pig farm of | nitrogen, storage at −80° C. |
| | Institute of Animal | |
| | Husbandry and Veterinary, | |
| | Hubei Academy of | |
| | Agricultural Sciences | |
| pGL3-basic, | All purchased from | pGL3-basic and pGL3-promoter |
| pGL3-promoter, | Promega(USA) | express firefly luciferase, |
| pRL-TK reporter | | while pRL-TK reporter vector |
| vector | | expresses renilla luciferase |
| | | reporter gene |
| High fidelity | Toyobo(Japan) | Amplification of 5′ |
| polymerase KOD plus | | untranslated region of pig |
| | | myostatin gene locus |
| Taq DNA polymerase, | Shanghai Biocolor | Identification of positive |
| dNTP | BioScience & Technology | transformants |
| | Company | |
| MSTN-5F upstream | Synthesized by Shanghai | TTCA ACGCGTTCACGACAACGCCGGAT |
| primer (5′→3′) | Invitrogen Biotechnology | CCTTAACCC (MluI) |
| | Company | (SEQ ID NO. 5) |
| MSTN-5R downstream | Synthesized by Shanghai | CATG CTCGAGCGCCAAGCAAAATTTTA |
| primer (5′→3′) | Invitrogen Biotechnology | ATGCC (XhoI) |
| | Company | (SEQ ID NO. 6) |
| Sequencing primer | Synthesized by Shanghai | CTTTATGTTTTTGGCGTCTTCC |
| GLprimer2 (5′→3′) | Invitrogen Biotechnology | |
| | Company | |
| XhoI, MluI restriction | Takara(Japan) | Digested DNA fragments |
| endonucleases | | |
| DNA Gel Extraction Kit | TransGen Biotech, Inc., | Gel excision and PCR |
| | Beijing | product recovery |
| Ultrapure Plasmid | TransGen Biotech, Inc., | Preparation of endotoxin- |
| Extraction Kit | Beijing Fermentas | free Plasmid Ligation of |
| T4 DNA Ligase | | vector with inseted fragments |
| SeaKem agarose | FMC, US | Electrophoresis detection |
| | | and analysis |
| ethidium bromide | Sigma | Nucleic acid staining |
| Tris-Phenol, | China National Medicines | Nucleic acid extraction |
| chloroform, isoamyl | Corporation Ltd. | |
| alcohol, anhydrous | (analytical grade) | |
| ethanol, sodium acetate | | |
| 1 kb DNA marker | Guangzhou Dongsheng | Identification for sizes of |
| | Biotech Co., Ltd. | nucleic acid fragments |
| Protease K | Sigma | Remove of proteins during |
| | | nucleic acid extraction |
| Rnase | Amresco | Remove of RNA |
| Amp | Amresco | Transformation and screen |

### 1) Pig Genomic DNA Extraction

In vitro muscle tissue sample of pig ear, 0.1 g, was taken from piglets, washed and shredded, from which the pig genomic DNA was isolated.

### 2) PCR Amplification for Fragments of the 5′ Untranslated Region of Pig Myostatin Gene Locus

The 5′ untranslated region of pig myostatin gene locus was amplified with specific primers (MSTN-5F and MSTN-5R) carrying pre-determined enzyme digestion sites using the above resulting genomic DNA as a template. After the reaction finished, PCR product was detected by a 1% agarose gel electrophoresis. Experimental results are shown in FIG. 1, M is a 1 kb DNA marker having the following lengths in the order of size: 0.5 kb, 1 kb, 1.5 kb, 2 kb, 3 kb, 4 kb, 5 kb, 6 kb, 8 kb and 10 kb. The PCR amplification gave a fragment of 3778 bp.

The PCR product was sent for sequencing in BGI-Shenzhen using Glprimer2 as the sequencing primer. Sequencing results showed that this PCR product had nucleotides as set forth in SEQ ID NO. 1 in the sequence listing. The PCR product was designated as DNA fragment A, i.e., sequence of the 5′ untranslated region of pig myostatin gene locus. As can be seen from the general features of transcription of eukaryotes genes, positions 3646-3650 starting from the 5′ terminus of this sequence is an element necessary for transcription of the 5′ terminus, a CAAT cassette (5′ terminal CAAT cassette), positions 3687-3693 starting from the 5′ terminus is an element necessary for transcription of upstream, a TATA box, and positions 3710-3716 starting from the 5′ terminus is an element necessary for transcription of downstream, a TATA box.

SEQ ID NO. 1 may also be artificially synthesized.

### 3) Cloning and Identification of the 5′ Untranslated Region of Pig Myostatin Gene Locus

The PCR product resulting from 2) was digested with MluI and XhoI to give a fragment, which was ligated with a fragment produced by digestion of the reporter vector pGL3-basic with the same enzymes using the following system. The ligation product was then transformed into Escherichia coli DH5α, and cultured at 37° C. overnight to give a transformant. Colony PCR was conducted to detect the above resulting transformants using MSTN-5F and MSTN-5R as the primers. A recombinant giving an amplification product of 3778 bp is a positive plasmid, which was then sent for sequencing. The result showed that this plasmid, designated as pGL3-5′MSTN, was obtained by inserting SEQ ID NO. 1 in the sequence listing into pGL3-baisc at the MluI and XhoI enzyme digestion sites. SEQ ID NO. 1 was inserted upstream of firefly luciferase in pGL3-baisc.

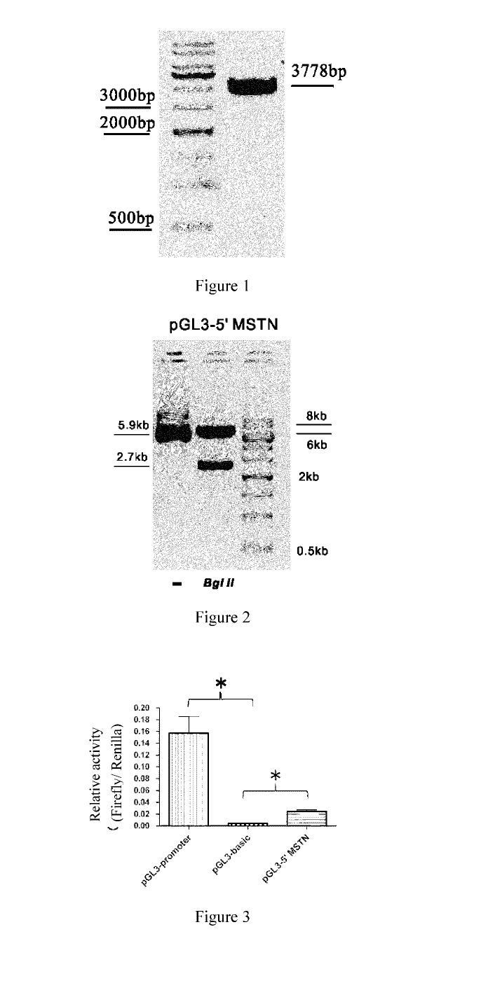

There is a naturally-occurring BglII digestion site inside the cloned 5′ untranslated region of pig myostatin gene locus, and another BglII digestion site at a multiple cloning site of the basic vector, pGL3-basic. A single digestion was conducted on pGL3-5′MSTN with the restriction endonuclease, BglII. The results are shown in FIG. 2, wherein “−” denotes undigested pGL3-5′MSTN, and “BglII” denotes BglII digested pGL3-5′MSTN. As can be seen, this vector gave a fragment of 2.7 kb after BglII digestion, which was completely consistent with theoretical analysis, indicating that this vector harbors the sequence of the 5′ untranslated region of pig myostatin gene.

### 2. Identification of the Transcriptional Activity of the 5′ Untranslated Region of Pig Myostatin Gene Locus

TABLE 2
Experimental materials

| Main materials | Sources | Instructions |
|---|---|---|
| Lipofectamine2000 | Invitrogen | Cell transfection |
| Mouse myoblast C2C12 | ATCC cell line, code | Myocyte model |
| | number: CRL-1772 | |
| Dual Luciferase | Promega | Detection for enzyme activity |
| Assay Kit | | of reporter gene |
| DLR^{M} Assay | | |
| Ultrapure | Plasmid TransGen Biotech, | Preparation of endotoxin-free |
| Extraction Kit | Inc., Beijing | Plasmid for cell transfection |
| Opti-MEM | Gibco | cell transfection auxiliary |
| | | reagent |
| 24-well cell culture plate | Corning | Cell culture |

### 1). Cell Transfection

4×10⁴ C2C12 cells were seeded onto a 24-well plate one day before transfection, and growed overnight. Subsequently, transfection complexes were formulated according to the system set forth in the table below, dropped into the 24-well plate, which was supplemented with 400 μl of complete medium afterwards, and cells were cultured under the condition of 37° C., 5% CO₂. The cells were collected after being transfected for 48h. Then, luciferase activity was determined based on the instructions of the DLR^{M} Assay kit from Promega Company.

The transfection system is shown as below:

TABLE 3
Transfection system

| Plasmid | Amount | Opti-MEM | Lipofectamine2000 |
|---|---|---|---|
| pGL3-basic | 100 ng | 100 μl | 0.5 μl |
| PGL3-promoter | 100 ng | 100 μl | 0.5 μl |
| pGL3-5′MSTN | 100 ng | 100 μl | 0.5 μl |
| pRL-TK(internal control) | 100 ng | 100 μl | 0.5 μl |

The experiments were performed on three biological replicates with the average values taken as the result. The results are shown in Table 4 and FIG. 3.

TABLE 4
Absolute value and relative ratio of enzyme activities of the reporter vector

| | Firefly luciferase | Renilla luciferase | Enzyme activity |
|---|---|---|---|
| | (Fluc) | (Rluc) | ratio (Fluc/Rluc) |
| pGL3-promoter | 25823 | 208944 | 0.1236 |
| | 85796 | 402409 | 0.2132 |
| | 51815 | 383733 | 0.135 |
| pGL3-basic | 1325 | 249998 | 0.0053 |
| | 956 | 246479 | 0.0039 |
| | 867 | 222630 | 0.0039 |
| pGL3-5′MSTN | 8511 | 438967 | 0.0194 |
| | 12605 | 447943 | 0.0281 |
| | 9158 | 354874 | 0.0258 |

As shown in Table 4 and FIG. 3, the cloned 5′ untranslated region of pig myostatin gene locus of the present invention was demonstrated to actually have certain transcriptional activity by luciferase reporter gene assay, because it contains a transcription initiation element necessary for transcription of eukaryotes; the activity thereof is weaker as compared to SV40 promoter of the positive control plasmid, pGL3-promoter, but still stronger as compared to the negative control plasmid, pGL3-basic, which is free of a promoter. * denotes p<0.05, indicating a significant difference. On one hand, it suggests that the 5′ untranslated region of pig myostatin gene locus obtained by the present invention harbors a promoter element of pig myostatin gene; on the other hand, it indicates that this untranslated region may be served as a target site for gene targeting so as to be used in the transcription and expression of inactive pig myostatin gene; moreover, this sequence may also act as a homologous arm for gene targeting, which drives in situ expression of foreign genes when functioning in homologous recombination. The 5′ untranslated region is a promoter.

### II. Cloning and Activity Identification of the 3′ Untranslated Region of Pig Myostatin Gene Locus

### 1. Cloning of the 3′ Untranslated Region of Pig Myostatin Gene Locus

TABLE 5
Experimental materials

| Main materials | Sources | Instructions |
|---|---|---|
| MSTN-UTR-F | Synthesized by Shanghai | TTCA GTTAACGGTTCATTACTTCCTAAAA |
| upstream primer | Invitrogen Biotechnology | CATGG (HpaI) |
| (5′→3′) | Company | |
| MSTN-UTR-R | Synthesized by Shanghai | CATG GTCGACGTTTCTACACATTAGATGT |
| downstream | Invitrogen Biotechnology | AAG (SalI) |
| primer (5′→3′) | Company | |
| Sequencing | Synthesized by Shanghai | GGAAAGATCGCCGTGTAAT |
| primer UTR-S | Invitrogen Biotechnology | |
| | Company | |
| HpaI, SalI | Takara(Japan) | digested DNA fragments |
| restriction | | |
| endonuclease | | |

### 1) Extraction of Pig Genomic DNA

Genomic DNA was isolated from ear muscle tissue in vitro of a piglet.

### 2) PCR Amplification for Fragments of the 3′ Untranslated Region of Pig Myostatin Gene Locus

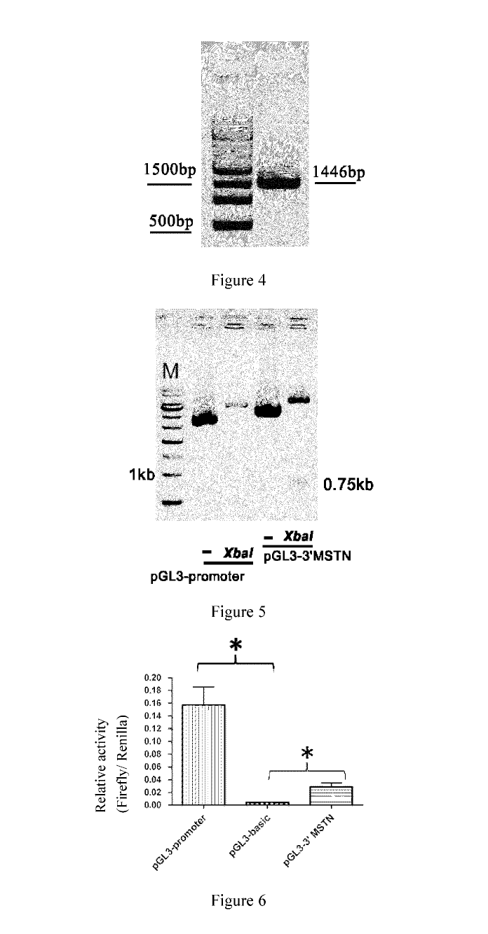

The 3′ untranslated region of pig myostatin gene locus was amplified by specific primers (MSTN-UTR-F and MSTN-UTR-R) carrying pre-determined enzyme restriction sites using the above resulting genomic DNA as a template. After the reaction finished, PCR product was detected by a 1% agarose gel electrophoresis. The results are shown in FIG. 4, M is a 1 kb DNA marker having the following lengths in the order of size: 0.5 kb, 1 kb, 1.5 kb, 2 kb, 3 kb, 4 kb, 5 kb, 6 kb, 8 kb and 10 kb. The PCR amplification gave a fragment of 1446 bp.

The PCR product was sent for sequencing in BGI-Shenzhen using UTR-S as the sequencing primer. Sequencing results showed that this PCR product had nucleotides as set forth in SEQ ID NO. 3 in the sequence listing. The PCR product was designated as DNA fragment C, i.e., sequence of the 3′ untranslated region of pig myostatin gene locus. As can be seen from the general features of transcription termination of eukaryotes genes, positions 183-188, 597-602, 921-926 and 1282-1287 starting from the 5′ terminus of this sequence are AATAAA signal sequences necessary for the polyadenylation of the messenger mRNA of pig myostatin gene.

SEQ ID NO. 3 may also be artificially synthesized.

### 3) Cloning and Identification of the 3′ Untranslated Region of Pig Myostatin Gene Locus

The PCR product resulting from the above 2) was digested with HpaI and SalI to give a fragment, which was ligated with a fragment produced by digestion of the reporter vector, pGL3-promoter, with the same enzymes using the following system. The E. coli DH5α was then transformed with the ligation product, and cultured at 37° C. overnight to give a transformant. Colony PCR was conducted to detect the above resulting transformants with MSTN-UTR-F and MSTN-UTR-R as the primers. A recombinant giving an amplification product of 1446 bp is the positive plasmid, which was then sent for sequencing. The result showed that this plasmid, designated as pGL3-3′MSTN, was obtained by inserting SEQ ID NO. 3 in the sequence listing into pGL3-promoter between the HpaI and SalI enzyme digestion sites. SEQ ID NO. 3 was inserted downstream of firefly luciferase in pGL3-promoter.

There are a naturally-occurring XbaI enzyme digestion site inside the cloned 3′ untranslated region of pig myostatin gene locus, and another XbaI enzyme digestion site at the terminus of the renilla luciferase gene in the basic vector, pGL3-promoter. A single enzyme digestion was conducted on pGL3-3′MSTN with the restriction endonuclease, XbaI. The results are shown in FIG. 5, wherein “−” denotes the plasmid, and “XbaI” denotes the digested plasmid. As can be seen, pGL3-3′MSTN gave a fragment of 0.75 kb after XbaI digestion, which was completely consistent with theoretical analysis, indicating that this vector harbors the sequence of the 3′ untranslated region of pig myostatin gene.

### 2. Identification of the Transcriptional Activity of the 3′ Untranslated Region of Pig Myostatin Gene Locus

### 1). Cell Transfection

4×10⁴ C2C12 cells were seeded onto a 24-well plate one day before transfection, and growed overnight. Subsequently, transfection complex were formulated according to the system set forth in the table below, dropped into the 24-well plate, which was supplemented with 400 μl of complete medium afterwards, and the cells were cultured under the condition of 37° C., 5% CO₂. The cells were collected after being transfected for 48h. Then, luciferase activity was determined based on the instructions of the DLR^{M} Assay kit from Promega Company.

The transfection system was the same as that presented in the above Talbe 3. The experiments were performed on three biological replicates with the average values taken as the result. The results are shown in Table 6 and FIG. 6.

TABLE 6
Absolute value and relative ratio of enzyme activities of the reporter vector

| | Firefly luciferase | Renilla luciferase | Enzyme activity |
|---|---|---|---|
| | (Fluc) | (Rluc) | ratio (Fluc/Rluc) |
| pGL3-promoter | 25823 | 208944 | 0.1236 |
| | 85796 | 402409 | 0.2132 |
| | 51815 | 383733 | 0.135 |
| pGL3-basic | 1325 | 249998 | 0.0053 |
| | 956 | 246479 | 0.0039 |
| | 867 | 222630 | 0.0039 |
| pGL3-3′MSTN | 17178 | 447038 | 0.0384 |
| | 10927 | 366709 | 0.0298 |
| | 2010 | 114086 | 0.0176 |

As shown in Table 6 and FIG. 6, the cloned 3′ untranslated region of pig myostatin gene locus of the present invention was demonstrated to actually have certain transcriptional termination activity by luciferase reporter gene assay, because it contains a transcription termination element necessary for the transcription of eukaryotes. * denotes p<0.05, indicating a significant difference. On one hand, it suggests that the 3′ untranslated region of pig myostatin gene locus obtained by the present invention harbors a termination element of pig myostatin gene; on the other hand, it indicates that this untranslated region may be served as a target site for gene targeting so as to be used in the transcription and expression of inactive pig myostatin gene; moreover, this sequence may also act as a homologous arm for gene targeting, which functions to terminate the transcription of foreign genes and facilitate translation while functioning in homologous recombination. The 3′ untranslated region is a terminator.

### Example 2

### Isolation of Full Sequence of Pig Myostatin Gene Locus and Studies on the Functions Thereof

### I. Isolation of Full Sequence of Pig Myostatin Gene Locus

### 1. Experimental Materials

TABLE 7
Experimental materials

| Main materials | Sources | Instructions |
|---|---|---|
| pIC19 cloning vector | Purchased from Takara | Prokaryotic expression |
| | | vector, usually used for |
| | | gene cloning |
| pIRES2-EGFP | Purchased from Invitrogen | Expression vector of green |
| | | fluorescent protein EGFP |
| High fidelity | Toyobo(Japan) | Amplification of DNA |
| polymerase KOD plus | | fragments of pig myostatin |
| | | gene locus |
| Taq DNApolymerase, | Shanghai Biocolor | Identification of positive |
| dNTP | BioScience & Technology | transformants |
| | Company | |
| Upstream primer | Synthesized by Shanghai | ATCGGTACCATCATTAAACTTCTGAC |
| MSTN-5′F(5′→3′) of | Invitrogen Biotechnology | AAGCC |
| the 5′ untranslated | Company | (KpnI) |
| region | | |
| Downstream primer | Synthesized by Shanghai | ATCGGATCCGCCAAGCAAAATTTTAA |
| MSTN-5′R(5′→3′) of | Invitrogen Biotechnology | TGCC |
| the 5′ untranslated | Company | (BamHI) |
| region | | |
| Upstream primer | Synthesized by Shanghai | ATCGTCGACGGTTCATTACTTCCTAA |
| MSTN-3′F(5′→3′) of | Invitrogen Biotechnology | AACATGG |
| the 3′ untranslated | Company | (PstI) |
| region | | |
| Downstream primer | Synthesized by Shanghai | ATCAAGCTTGTTTCTACACATTAGAT |
| MSTN-3′R(5′→3′) of | Invitrogen Biotechnology | GTAAG |
| the 3′ untranslated | Company | (HindIII) |
| region | | |
| Upstream primer | Synthesized by Shanghai | ATCGGATCCTTACTCAAAAGCAAAAG |
| MSTN-F(5′→3′) of | Invitrogen Biotechnology | TAAAAGGA |
| pig myostatin gene | Company | (BamHI) |
| Upstream primer | Synthesized by Shanghai | ATCAAGCTTAAATATAAATCTCATGA |
| MSTN-R(5′→3′) of | Invitrogen Biotechnology | GCACCC |
| pig myostatin gene | Company | (PstI) |
| Upstream primer | Synthesized by Shanghai | ATCGGATCCACCATGGTGAGCAA |
| EGFP-F(5′→3′) of | Invitrogen Biotechnology | (BamHI) |
| green fluorescent | Company | |
| protein | | |
| Downstream primer | Synthesized by Shanghai | ATCGTCGACTTACTTGTACAGCT |
| EGFP-R(5′→3′) of | Invitrogen Biotechnology | (SalI) |
| green fluorescent | Company | |
| protein | | |
| Sequencing primer | Synthesized by Shanghai | CATTGTGGAGCAAGAGCC |
| MSTN-S1 (5′→3′) | Invitrogen Biotechnology | |
| | Company | |
| Sequencing primer | Synthesized by Shanghai | CTGTAGCATACTCCAGGCA |
| MSTN-S2 (5′→3′) | Invitrogen Biotechnology | |
| | Company | |
| BamHI, KpnI, PstI, | Takara(Japan) | Digestion of DNA fragments |
| HindIII, SalI | | |
| restriction | | |
| endonucleases | | |
| DL2000 DNA marker | Takara | Identification for sizes of |
| | | nucleic acid fragments |
| 1 kb DNA marker | Guangzhou Dongsheng | Identification for sizes of |
| | Biotech Co., Ltd. | nucleic acid fragments |
| Lamda DNA/Eco91I | Fermentas | Identification for sizes of |
| marker | | nucleic acid fragments |
| 1 kb plus DNA | Invitrogen | Identification for sizes of |
| marker | | nucleic acid fragments |

### 2. Experimental Methods

### 1) Obtaining of the 5′ Untranslated Region of Pig Myostatin Gene Locus, the Pig Myostatin Gene, and the 3′ Untranslated Region of Pig Myostatin Gene Locus

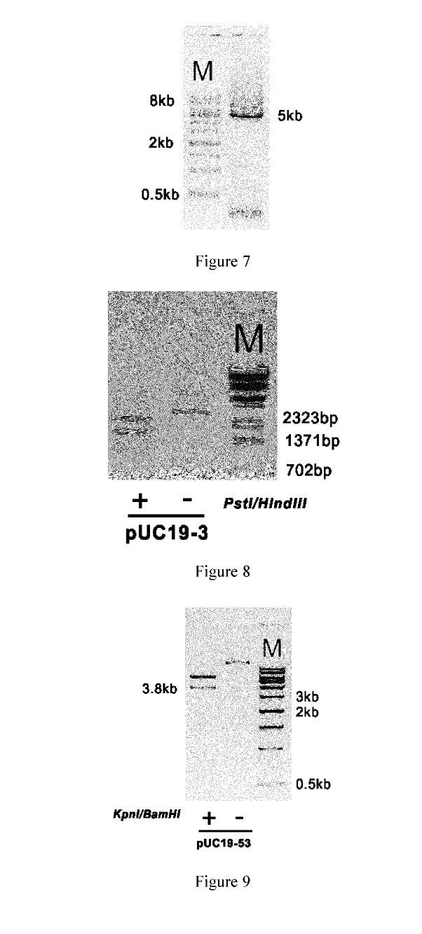

In vitro muscle tissue sample of pig ear was taken from piglets, from which genomic DNA was isolated. The above resulting genomic DNA was used as a template to conduct a PCR amplification with MSTN-F and MSTN-R as the primers. The results are shown in FIG. 7. The PCR product of 5 kb was designated as DNA fragment B, which, after sequencing, was shown to have a nucleotide sequence presented by SEQ ID NO. 2. This is completely consistent with theoretical analysis, indicating that the full sequence of the coding region of pig myostatin gene MSTN is obtained.

In vitro muscle tissue sample of pig ear was taken from piglets, from which genomic DNA was isolated. The above resulting genomic DNA was used as a template to conduct a PCR amplification with MSTN-5′F and MSTN-5′R as the primers. The resultant PCR product was designated as DNA fragment A, i.e., the 5′ untranslated region of pig myostatin gene locus (SEQ ID NO. 1, promoter);

The above resulting genomic DNA was used as a template to conduct a PCR amplification with MSTN-3′F and MSTN-3′R as the primers. The resultant PCR product was designated as DNA fragment C, i.e., the 3′ untranslated region of pig myostatin gene locus (SEQ ID NO. 3, terminator).

2) Insertion of the 3′ Untranslated Region into pUC19 Vector

The DNA fragment C of the 3′ untranslated region resulted from the above 1) was digested with PstI and HindIII, ligated with pUC19 vector digested with the same enzymes, and then, E. coli DH5α was transformed with the ligation product and cultured at 37° C. overnight to give a transformant. A colony PCR was performed to detect the above resultant transformants with MSTN-3′F and MSTN-3′R as the primers. A recombinant giving an amplification product of 1446 bp is a positive plasmid, on which a double enzyme digestion was conducted with PstI and HindIII. The results are shown in FIG. 8, wherein, “+” denotes the digested plasmid, and “−” denotes the plasmid. As can be seen from the results, a fragment of 1446 bp was produced, which is consistent with theoretical analysis. M is a Lamda DNA/Eco91I marker, with the following lengths in the order of size: 702 bp, 1264 bp, 1371 bp, 1929 bp, 2323 bp, 3675 bp, 4324 bp, 4822 bp, 6369 bp, 7242 bp and 14140 bp. After sequencing, this plasmid, designated as pUC19-3, is shown to be a vector obtained by inserting SEQ ID NO. 3 in the sequence listing into pUC19 at the digestion sites of PstI and HindIII.

3) Insertion of 5′ Untranslated Region into the pUC19-3 Vector

The DNA fragment A of the 5′ untranslated region resulted from the above 1) was digested with KpnI and BamHI, ligated with the pUC19-3 vector from step 2) digested with the same enzymes, and then, E. coli DH5α was transformed with the ligation product and cultured at 37° C. overnight to give a transformant. A colony PCR was conducted to detect the above resultant transformants with MSTN-5′F and MSTN-5′R as the primers. A recombinant capable of giving an amplification product of 3778 bp is a positive plasmid, on which a double enzyme digestion was conducted with BamHI and KpnI, generating a fragment of 3778 bp. The results are shown in FIG. 9, wherein, “+” denotes the digested plasmid and “−” denotes the plasmid, which is consistent with theoretical analysis. This plasmid is designated as pUC19-53. M is a 1 kb DNA marker, with the following lengths in the order of size: 0.5 kb, 1 kb, 1.5 kb, 2 kb, 3 kb, 4 kb, 5 kb, 6 kb, 8 kb and 10 kb.

4) Cloning of the Expression Cassette of Pig Myostatin Gene into a pUC19-53 Vector

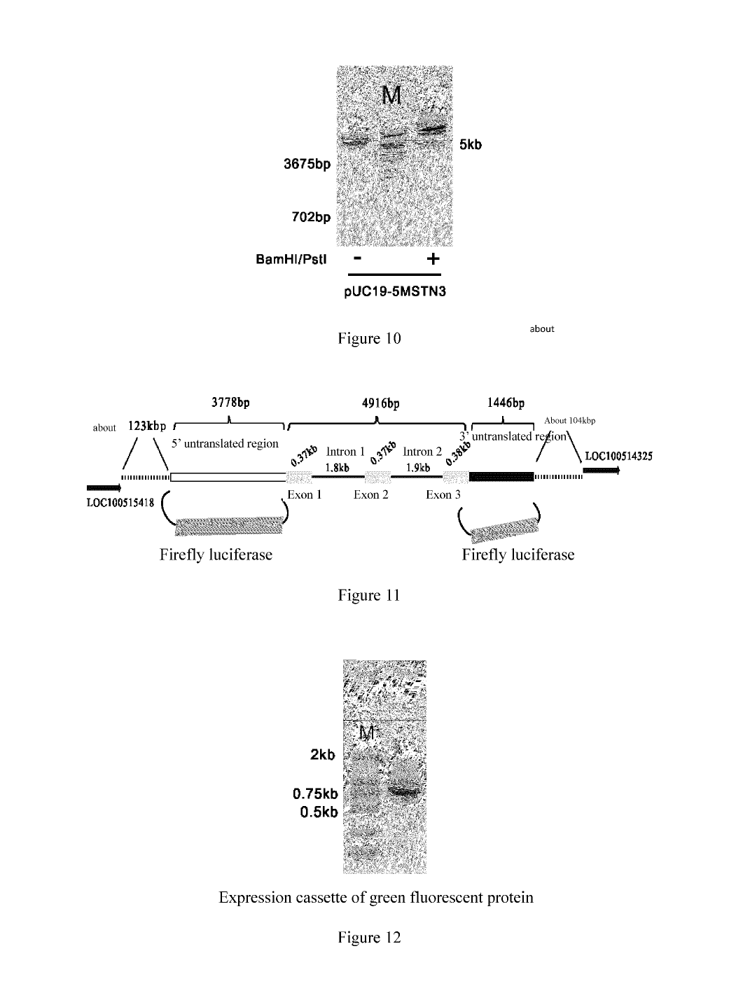

The expression cassette PCR product of pig myostatin gene from the above 1) (DNA fragment B) was digested with BamHI and PstI, ligated with fragments generated by digestion of pUC19-53 vector with the same enzymes, and then, E. coli DH5α was transformed with the ligation product and cultured at 37° C. overnight to give a transformant. A colony PCR was conducted to detect the above resultant transformants with MSTN-F and MSTN-R as the primers. A recombinant that is able to give an amplification product of 5 Kb is a positive plasmid, on which a double enzyme digestion was conducted with BamHI and PstI, generating a fragment of 5 kb from the vector. The results are shown in FIG. 10, wherein, “+” denotes the digested plasmid and “−” denotes the plasmid, which is consistent with theoretical analysis. This plasmid is designated as pUC19-SMSTN3. After sequencing, this plasmid was found to contain a DNA molecule consisting of the DNA fragment A of the 5′ untranslated region (SEQ ID NO. 1), the DNA fragment B of pig myostatin gene (SEQ ID NO. 2) and the DNA fragment C of the 3′ untranslated region (SEQ ID NO. 3) in the above order, that is, the pig myostatin gene locus; moreover, this DNA molecule was inserted into pUC19 between the KpnI and HindIII restriction sites.

FIG. 11 is a schematic view of the pig myostatin gene locus. As can be seen, the pig myostatin gene locus comprises three portions: the 5′ untranslated region, the myostatin gene encoding region, and the 3′ untranslated region. The pig myostatin gene locus is confirmed by the present invention to have a 5′ untranslated region of 3776 bp in length, which comprises all transcription initiation elements of the pig myostatin gene such as TATA box (two) and CAAT box (one), as well as MEF (myocyte enhancer factors) binding sequence and the like. The coding region of the myostatin gene is 3789 bp in full length comprising three exons and two introns. The exons have a length of 373 bp, 374 bp and 381 bp, respectively; and the introns have a length of 1809 bp and 1980 bp, respectively. The 3′ untranslated region, confirmed by the present invention to have a length of 1446 bp, comprises a polyadenylation signal (polyA signal) necessary for the translation of the messenger RNA (mRNA) of myostatin.

This gene locus may also be artificially synthesized.

II. In Vitro Expression of Foreign Genes with Pig Myostatin Gene Locus
1. Obtaining of the Reporter Vector pUC19-5EGFP3

### 1) Obtaining of the Expression Cassette of Green Fluorescent Protein

pIRES2-EGFP was used as a template to perform a PCR with EGFP-F and EGFP-R as the primers. The resultant PCR product was sequenced and detected by a 1% agarose gel electrophoresis. The results are shown in FIG. 12, wherein, M is a DL2000 DNA marker, with the following lengths in the order of size: 0.1 kb, 0.25 kb, 0.5 kb, 0.75 kb, 1 kb and 2 kb. The PCR amplification produced a fragment of the green fluorescent protein of 720 bp, which, after being sequenced, was shown to have a nucleotide sequence as presented by SEQ ID NO. 4 (expression cassette of the green fluorescent protein), and an amino acid sequence of which was presented by SEQ ID NO. 5.

2) Obtaining of the pUC19-5EGFP3

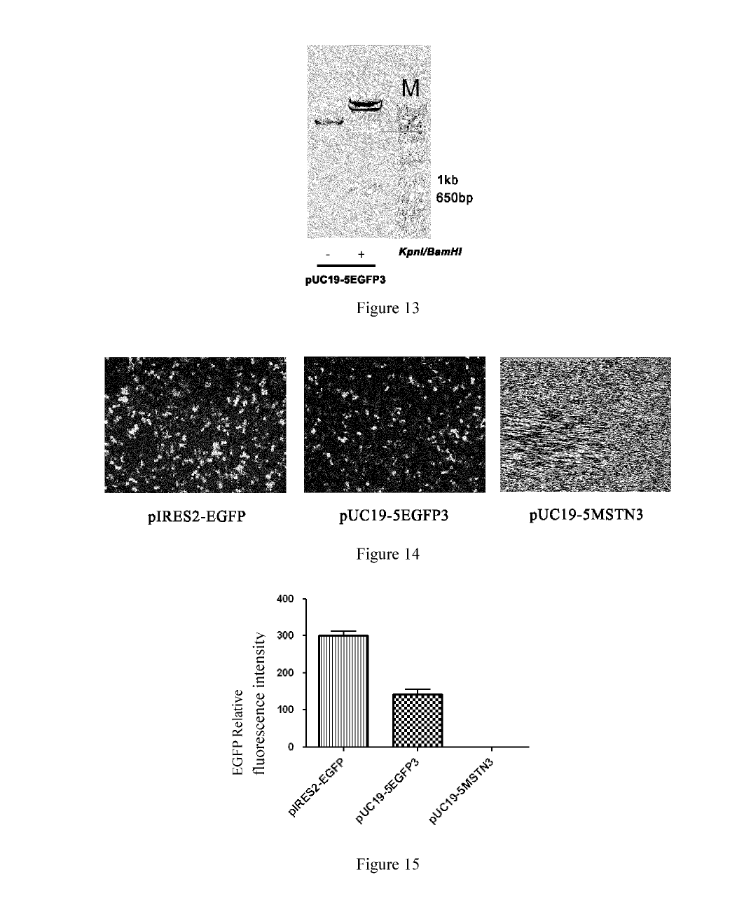

The expression cassette of the green fluorescent protein (SEQ ID NO. 4) from the above 1) was digested with BamHI and SalI, ligated with fragments produced by digestion of the pUC19-5MSTN3 vector from the above I with the same enzymes, and then, E. coli DH5α was transformed with the ligation product and cultured at 37° C. overnight to give a transformant. A colony PCR was conducted with EGFP-F and EGFP-R as the primers to detect the above resultant transformants. A recombinant that is able to give an amplification product of 720 bp is a positive plasmid, on which a double enzyme digestion was conducted with BamHI and SalI. The results are shown in FIG. 13, wherein, “+” denoted the digested plasmid and “−” denotes the plasmid. As can be seen, a fragment of 720 bp was generated from this vector after digestion, which is consistent with theoretical analysis. M is a 1 kb plus DNA marker, having various fragments in the lengths of: 100 bp, 200 bp, 300 bp, 400 bp, 500 bp, 650 bp, 850 bp, 1000 bp, 1650 bp, 2000 bp, 3000 bp, 4000 bp, 5000 bp, 6000 bp, 7000 bp, 8000 bp, 9000 bp, 10000 bp and 12000 bp, respectively. Afterwards, the positive plasmid was sent for sequencing, showing that this plasmid was obtained by inserting SEQ ID NO. 4 into pUC19-5MSTN3 at BamHI and SalI restriction sites and replacing MSTN (SEQ ID NO. 2). This plasmid is designated as pUC19-5EGFP3. That is, the promoter, the expression cassette of the green fluorescent protein and the terminator were inserted into pUC 19 at the KpnI and HindIII restriction sites.

2. Detection of the Expression of the Reporter Vector pUC19-5EGFP3

4×10⁴ C2C12 cells were seeded onto a 24-well plate one day before transfection, and growed overnight. Subsequently, transfection complexes were formulated according to the system set forth in the table below, dropped into the 24-well plate, which was supplemented with 400 μl of complete medium afterwards, and the cells were cultured under the condition of 37° C., 5% CO₂. After 24 hours, Leika microscope (German) was used to observe expressions of the green fluorescent protein.

The transfection system is as follows:

TABLE 8
Transfection system

| Plasmid | Amount | Opti-MEM | Lipofectamine2000 |
|---|---|---|---|
| pIRES2-EGFP | 100 ng | 100 μl | 0.5 μl |
| (positive control) | | | |
| pUC19-5MSTN3 | 100 ng | 100 μl | 0.5 μl |
| (negative control) | | | |
| pUC19-5EGFP3 | 100 ng | 100 μl | 0.5 μl |

The results are shown in FIG. 14. As can be seen, the positive control, pIRES2-EGFP, has a stronger expression of the green fluorescent protein, while the negative control, pUC19-5MSTN3, does not express the green fluorescent protein. After transfection of myocyte with pUC19-5EGFP3, this vector may be observed to have a stronger ability to express the green fluorescent protein.

The ImageJ (http://rsbweeb.nih.gov/ij/download.html) software was employed to analyze the fluorescence intensities of pIRES2-EGFP (positive control), pUC19-5MSTN3 (negative control) and pUC19-5EGFP3 of FIG. 14. As shown in FIG. 15 (with pUC19-5MSTN3 (negative control) as the basis), the results demonstrate that the pig myostatin gene locus provided by the present invention is able to express foreign genes under in vitro experimental conditions.

### INDUSTRIAL APPLICATION

The present invention provides a pig myostatin gene locus comprising a 3778 bp 5′ untranslated region upstream of the encoding region of the myostatin gene, a 4916 bp encoding region and a 1446 bp 3′ untranslated region downstream of the encoding region of the myostatin gene. The luciferase reporter gene assay demonstrates that the 5′ untranslated region of the pig myostatin gene locus has a transcription initiation activity, and the 3′ untranslated region has a transcription termination activity, the both and the coding region of the myostatin gene constitute an intact expression unit, i.e., a gene locus. Meanwhile, the present invention constructs a green fluorescent protein reporter vector based on this gene locus, indicating that this gene locus, under in vitro experimental conditions, is able to effectively start the expression of a foreign gene. This gene locus has the following utilities: (1) inserting a foreign gene into the coding region of myostatin gene of the gene locus, and regulating the expression thereof by the 5′ untranslated region and 3′ untranslated region to generate a corresponding recombinant plasmid, recombinant strain, transgenic cell line or transgenic animal; (2) inactivating the myostatin using the 5′ untranslated region or the 3′ untranslated region as the targeting site, or inactivating the myostatin while inserting a foreign gene, so as to generate a corresponding recombinant plasmid, recombinant strain, transgenic cell line or transgenic animal; (3) inactivating the myostatin with partial or whole sequence of any two of the 5′ untranslated region, the coding region and the 3′ untranslated region as the targeting site, or inactivating the myostatin while introducing a foreign gene, to generate a corresponding recombinant plasmid, recombinant strain, transgenic cell line or transgenic animal; (4) inactivating the myostatin with the whole pig myostatin gene locus as the targeting site, or inactivating the myostatin while introducing a foreign gene, to generate a corresponding recombinant plasmid, recombinant strain, transgenic cell line or transgenic animal. The present invention provides a reliable and valuable gene source in solving the problems such as unstable expression of a foreign gene in a transgenic pig, unpredictable nature of the position effect and the like.

1.-5. (canceled)

6. A terminator, which is a DNA molecule selected from the group consisting of (a) through (c) as follows:
(a) a DNA molecule as set forth in SEQ ID NO. 3 in the sequence listing;
(b) a DNA molecule, hybridizing with the DNA sequence as defined in (a) under stringent conditions and having a same function as the DNA sequence as defined in (a);
(c) a DNA molecule, having at least 70%, homology to the DNA molecule sequence as defined in (a) and having a same function as the DNA sequence as defined in (a).

7. (canceled)

8. The terminator according to claim 6, wherein the DNA molecule is as described in (c) and the DNA molecule has at least 75% homology to the DNA molecule sequence as defined in (a).

9. The terminator according to claim 6, wherein the DNA molecule is as described in (c) and the DNA molecule has at least 80% homology to the DNA molecule sequence as defined in (a).

10. The terminator according to claim 6, wherein the DNA molecule is as described in (c) and the DNA molecule has at least 85% homology to the DNA molecule sequence as defined in (a).

11. The terminator according to claim 6, wherein the DNA molecule is as described in (c) and the DNA molecule has at least 90% homology to the DNA molecule sequence as defined in (a).

12. The terminator according to claim 6, wherein the DNA molecule is as described in (c) and the DNA molecule has at least 95% homology to the DNA molecule sequence as defined in (a).

13. The terminator according to claim 6, wherein the DNA molecule is as described in (c) and the DNA molecule has at least 96% homology to the DNA molecule sequence as defined in (a).

14. The terminator according to claim 6, wherein the DNA molecule is as described in (c) and the DNA molecule has at least 97% homology to the DNA molecule sequence as defined in (a).

15. The terminator according to claim 6, wherein the DNA molecule is as described in (c) and the DNA molecule has at least 98% homology to the DNA molecule sequence as defined in (a).

16. The terminator according to claim 6, wherein the DNA molecule is as described in (c) and the DNA molecule has at least 99% homology to the DNA molecule sequence as defined in (a).

17. An expression cassette comprising a promoter, a foreign gene and a terminator according to claim 6.

18. The expression cassette according to claim 17, wherein the promoter is a DNA molecule selected from the group consisting of (i) through (iv) as follows:
(i) nucleotides at positions 2642-3778 starting from the 5′ end of SEQ ID NO. 1 in the sequence listing;
(ii) nucleotides as set forth in SEQ ID NO. 1 in the sequence listing;
(iii) a DNA molecule hybridizing to the DNA sequence as defined in (i) or (ii) under stringent conditions and having a same function as the DNA sequence as defined in (i) or (ii);
(iv) a DNA molecule having at least 70% homology with the DNA molecule sequence as defined in (i) or (ii) and having a same function as the DNA sequence as defined in (i) or (ii).

19. The expression cassette according to claim 18, wherein the promoter is a DNA molecule as defined in (iv), which has at least 75% homology with the DNA molecule sequence as defined in (i) or (ii).

20. The expression cassette according to claim 18, wherein the promoter is a DNA molecule as defined in (iv), which has at least 80% homology with the DNA molecule sequence as defined in (i) or (ii).

21. The expression cassette according to claim 18, wherein the promoter is a DNA molecule as defined in (iv), which has at least 85% homology with the DNA molecule sequence as defined in (i) or (ii).

22. The expression cassette according to claim 18, wherein the promoter is a DNA molecule as defined in (iv), which has at least 90% homology with the DNA molecule sequence as defined in (i) or (ii).

23. The expression cassette according to claim 18, wherein the promoter is a DNA molecule as defined in (iv), which has at least 95% homology with the DNA molecule sequence as defined in (i) or (ii).

24. The expression cassette according to claim 18, wherein the promoter is a DNA molecule as defined in (iv), which has at least 96% homology with the DNA molecule sequence as defined in (i) or (ii).

25. The expression cassette according to claim 18, wherein the promoter is a DNA molecule as defined in (iv), which has at least 97% homology with the DNA molecule sequence as defined in (i) or (ii).

26. The expression cassette according to claim 18, wherein the promoter is a DNA molecule as defined in (iv), which has at least 98% homology with the DNA molecule sequence as defined in (i) or (ii).

27. The expression cassette according to claim 18, wherein the promoter is a DNA molecule as defined in (iv), which has at least 99% homology with the DNA molecule sequence as defined in (i) or (ii).

28. The expression cassette according to claim 17, wherein the terminator comprises a nucleotide sequence as set forth in SEQ ID NO. 3 in the sequence listing.

29. The expression cassette of claim 17, wherein
the foreign gene is a pig myostatin gene as set forth in SEQ ID NO. 2 in the sequence listing or a green fluorescent protein-encoding gene as set forth in SEQ ID NO. 5 in the sequence listing.

30. A recombinant vector comprising the expression cassette of claim 17.

31. A recombinant strain comprising the expression cassette of claim 17.

32. A transgenic cell line comprising the expression cassette of claim 17.

33. A transgenic animal embryo or a transgenic animal comprising the expression cassette of claim 17.

34. The recombinant vector according to claim 30, wherein the recombinant vector is a recombinant vector obtained by inserting the expression cassette into pUC19 vector at a multiple cloning site.

35. A transgenic cell line obtained by introducing the recombinant vector of claim 30 into a host cell.

36. The transgenic cell line of claim 35, wherein the host cell is a C2C12 cell.

37. A method of expressing a foreign gene in a cell or animal comprising:
introducing a recombinant vector comprising the expression cassette of claim 17 into the cell or animal.
